(19) United States
(12) Patent Application Publication
(10) Pub. No.: US 2013/0102872 A1
(43) Pub. Date: Apr. 25, 2013

(54) ELECTRODE DEVICE FOR MEASURING IMPEDANCE OF HUMAN BODY AND APPARATUS COMPRISING THE SAME

(71) Applicant: Moon Seo Park, Incheon (KR)

(72) Inventor: Moon Seo Park, Incheon (KR)

(21) Appl. No.: 13/478,007

(22) Filed: May 22, 2012

Related U.S. Application Data
(63) Continuation of application No. PCT/KR2009/006953, filed on Nov. 25, 2009.

(30) Foreign Application Priority Data
Nov. 23, 2009 (KR) 10-2009-0113118

(51) Int. Cl. A61B 5/053

(52) U.S. Cl. CPC A61B 5/0531
USPC 600/393; 600/372; 600/395

(57) ABSTRACT

The present invention relates to an electrode device for measuring impedance within a human body, and to an apparatus using the same for measuring impedance within a human body and performing acupuncture treatment using the measured impedance, which can automatically and precisely determine locations of meridians within the human body, form a three-dimensional image of the determined locations, and enable acupuncture treatment to be performed precisely. The electrode device for measuring impedance within a human body comprises: a cylindrical housing member having a guide rod mounted to an open side thereof; a cylindrical electrode member configured to be pressed against the skin of a human body and moved back and forth through the open side along the guide rod; and a resilient member interposed between the cylindrical electrode member and the cylindrical housing member to resiliently move the cylindrical electrode member back and forth along the guide rod.

### CROSS-REFERENCE TO RELATED APPLICATION

This is a continuation application of PCT/KR2009/006953 filed Nov. 25, 2009, which claims the benefit of Korean Application No. 10-2009-0113118 filed Nov. 23, 2009, the entire contents of which applications are incorporated herein by reference.

### BACKGROUND

(a) Technical Field

The present disclosure relates to an apparatus for measuring impedance of a subject including human and treating certain conditions of the subject. More particularly, it relates to an apparatus for measuring impedance of a human body and treating a certain condition of a human body, which can precisely and automatically locate acupuncture points of a human body, display the location as a three-dimensional image, and facilitate acupuncture treatment using the located/displayed actupuncture points.

(b) Background Art

Acupuncture treats certain conditions of a human body (e.g., brain) by physically (e.g., mechanically or electrically) stimulating acupuncture points of the human body to change certain functions of the human body. The acupuncture's treating effects vary depending on whether the acupuncture points are precisely located. Practitioners locate the acupuncture points by their past experience and personal technique.

Electrical Impedance Tomography (EIT) was recently proposed and has been used as a medical imaging technique for some reasons. First, hardware systems using EIT are not expensive. Second, EIT is non-destructive and safe to a subject (e.g., human body). Third, temporal resolution provided by EIT is greater than those provided by X-ray or MRI while the spatial resolution of retrieved images is lower.

Acupuncture points of a human body are two-dimensional anatomically. Different human bodies have different distribution patterns of nerve tissues and soft tissues. Locating acupuncture points of a human body thus tends to rely on medical professionals' experience. EIT analyzes acupuncture points of a human body from the perspective of engineering.

According to EIT, a few milivolts of electric current at 10 to 100 kHz are allowed to flow through a human body and a resistance of the human body in response to the electric current is measured. Acupuncture points of a human body have an impedance lower than that of tissues surrounding the acupuncture points. To find out the electrical characteristics of cross-sections of a human body, a plurality of electrodes are attached to a human body, a certain level of electric current is allowed to flow through the human body in a predetermined order, resistance data in response to the electric current is measured, and the resistance data is displayed as an image.

A prior art electrical impedance tomography is described with reference to the drawings. FIGS. 1 to 4 show the principle of imaging resistance data of a human body in the prior art electrical impedance tomography.

A plurality of electrodes are attached to a human body. Electric current is allowed to flow through the human body sequentially. Resulting resistance data is measured and displayed as an image. As shown in FIG. 1, for example, 2×2 of input electrodes (S, s) and receiving electrodes (R, r) are attached to a human body, electric current is allowed to flow, and resulting resistance is measured.

In detail, as shown in FIG. 1, parallel input electrodes (S1, S2), parallel receiving electrodes (R1, R2), vertical input electrodes (s1, s2), and vertical receiving electrodes (r1, r2) are attached to a subject. As shown in FIG. 2, electric current is then allowed to flow from the parallel input electrodes (S1, S2) to the parallel receiving electrodes (R1, R2) and impedance in the horizontal direction is measured. As shown in FIG. 3, electric current is allowed to flow from the vertical input electrodes (s1, s2) to the vertical receiving electrodes (r1, r2) and impedance in the vertical direction is measured. As a result, as shown in FIG. 4, distribution of impedances of a human body can be estimated by reverse non-linear data processing.

Typically, an EIT apparatus has cylindrical electrodes that are designed to be attached to the skin of a human body by, for example, surrounding the skin (e.g., ankle, wrist, and the like). After the electrodes are attached to the skin, electrical current is allowed to flow through the human body sequentially and resulting resistances are measured. For example, the measured vertical and horizontal resistance values are the sum of all resistances of the body. Distribution of resistance data with regard to a certain cross-section of the body can be detected. Alternatively, from a known resistance distribution data, voltage distribution can be calculated according to the strength of the current, thereby being able to display an equipotential line.

The prior art EIT apparatus, however, has the following problems. First, as skeletons of a human body are imaged, it takes a long time to perform data processing. Second, depending on how the electrodes are attached to a human body, resolution of the image of acupuncture points can be significantly low. Third, although acupuncture points can be real-time detected, acupunctural treatment cannot be performed using the detected points.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

To solve the above-described problems, the present invention provides an electrode device for measuring impedances of a human body and an apparatus comprising the electrode device. The electrode device and the apparatus can automatically and precisely locate acupuncture points (meridians) of a human body, display the points as a three-dimensional image, and allow a precise acupuncture treatment to be performed at a desired location.

In one aspect, the present invention provides an electrode device for measuring impedances of a human body, comprising: a cylindrical housing member having a guide rod mounted to an open side thereof; a cylindrical electrode member configured to be pressed against the skin of a human body and moved back and forth through the open side along the guide rod; and a resilient member interposed between the cylindrical electrode member and the cylindrical housing member to resiliently move the cylindrical electrode member back and forth along the guide rod.

In some embodiments, the cylindrical electrode member is an electrode made of a conductive material or an electrode coated with a conductive material. The conductive material may be, e.g., gold.

In some embodiments, a protruding portion is formed at the open end of the cylindrical electrode member such that engagement between the cylindrical electrode member and the cylindrical housing member is facilitated.

In some embodiments, the cylindrical housing member is made of a conductive material or an electrode coated with a conductive material.

In some embodiments, a first protruding portion is formed at the open side of the cylindrical housing member and a second protruding portion is formed at the other side of the cylindrical housing member.

In some embodiments, the resilient member is made of a conductive material. The resilient member may be a spring, for example.

In another aspect, the present invention provides an apparatus for measuring impedance of a human body, which comprises: a base plate having a plurality of electrode holes formed in a lattice structure and a plurality of a first needle holes formed in a lattice structure such that the electrode holes and the first needle holes are arranged alternately; a plurality of electrodes mounted to the electrode holes; and first and second electrical lines connected to the electrodes.

In some embodiments, the distance between two adjacent electrode holes and the distance between two adjacent first needle holes are set in a range of 5 mm to 20 mm.

In some embodiments, the apparatus may further comprise a cover configured to be engaged with the base plate to protect the electrodes and the first and second electrical lines. Preferably, the cover may include a plurality of second needle holes arranged such that the respective second needle holes can be positioned over the respective first needle holes of the base plate when the cover is engaged with the base plate. Preferably, the outer width of the second needle holes may be designed to be greater than the inner width thereof.

In some embodiments, the cover is provided with at least one identifying member. Preferably, the cover may be made of silicone.

In some embodiments, the cover may be shaped to be a hemisphere or a cylinder. Preferably, the cover may further comprise a plurality of guide hole members each connecting the first needle holes and the second needle holes.

In some embodiments, the first electrical lines are formed between the respective electrodes and the respective first needle holes in the direction of X-axis and the second electrical lines are formed between the respective electrodes and the respective first needle holes in the direction of Y-axis.

In some embodiments, the first electrical lines are connected to input electrodes and the second electrical lines are connected to receiving electrodes.

In another aspect, the present inventions provides an apparatus for measuring impedances of a human body, comprising: a circular base plate including a plurality of electrode hoes and a plurality of first needle holes, the electrode holes and the first needle holes being formed eccentrically and alternately; a plurality of electrodes mounted to the electrode holes; and first and second electrical lines connected to the electrodes. Preferably, the first electrical lines and the second electrical lines may be connected to the electrodes alternately.

In still another aspect, the present invention provides an apparatus for measuring impedances of a human body, comprising: a plurality of base plates provided radially with a predetermined distance between two adjacent base plates, each of which base plates has a plurality of electrode holes and a plurality of first needle holes, the electrode holes and the first needle holes being mounted alternately; a plurality of electrodes mounted to the electrode holes; and first and second electrical lines connected to the electrode holes alternately. Preferably, the first electrical lines may be connected to input electrodes and the second electrical lines are connected to receiving electrodes.

In yet another aspect, the present invention provides an apparatus for measuring impedances of a human body, comprising: a plurality of base plates provided radially with a predetermined distance between two adjacent base plates, each of which base plates has a plurality of electrode holes and a plurality of first needle holes, the electrode holes and the first needle holes being mounted alternately with respect to the center of base plates; a plurality of electrode devices mounted to the electrode holes; and first and second electrical lines connected to the electrode holes alternately. Preferably, the first electrical lines may be connected to input electrodes and the second electrical lines are connected to receiving electrodes.

In a further aspect, the present invention provides an apparatus for measuring impedances of a human body, comprising: a base plate having a plurality of radial holes provided radially with a predetermined distance between two adjacent holes, the base plate having a plurality of electrode holes and a plurality of first needle holes between two adjacent radial holes; a plurality of electrode devices mounted to the electrode holes; and first and second electrical lines connected to the electrode devices alternately, wherein the electrode holes and the first needle holes are mounted alternately. Preferably, the first electrical lines may be connected to input electrodes and the second electrical lines are connected to receiving electrodes.

The present invention provides advantageous effects including the following. First, the data about the location of acupuncture points can be easily and quickly obtained. Second, acupuncture points can be located precisely regardless of curvature of the skin of a human body. Third, acupuncture points can be located and displayed without decrease in resolution regardless of the skin to be contacted by the apparatus. Fourth, the data about acupuncture points can be displayed as a three-dimensional image in combination with CT or MRI devices. Fifth, acupuncture points can be located automatically and acupuncture treatment can be performed according to the located points, which allow a user even without sufficient experience to use the apparatus.

The above and other features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIGS. 1 to 4 illustrate the principle of displaying resistance of a human body as an image according to a conventional electrical impedance tomography,

FIG. 5 is a cross-sectional view showing a dissembled state of an electrode device for measuring impedance of a human body according to the first embodiment of the present invention,

FIG. 6 is a cross-sectional view showing an assembled state of the electrode device of FIG. 5,

FIG. 7 is a cross-sectional view showing an example of application of the electrode device of FIG. 5,

FIG. 8 is a perspective view of an apparatus for measuring impedance of a human body according to the first embodiment of the present invention,

FIG. 9 is a perspective view of the bottom of the apparatus of FIG. 8,

FIG. 10 is a perspective view showing the apparatus of FIG. 8 comprising an an exemplary cover,

FIG. 11 is a perspective view showing the bottom of the the apparatus of FIG. 8 comprising an exemplary cover,

FIG. 12 is an enlarged view showing a hole of the cover of FIGS. 10 and 11,

FIG. 13 is a perspective view showing the apparatus of FIG. 8 comprising another exemplary cover,

FIG. 14 is a perspective view showing the apparatus of FIG. 8 comprising a still another exemplary cover,

FIG. 15 is an enlarged view showing a guide hole member of the covers of FIGS. 13 and 14,

FIGS. 16 and 17 illustrate that an apparatus according to the present invention has flexibility,

FIG. 18 is a perspective view showing the apparatus of FIG. 8 comprising a still further exemplary cover,

FIG. 19 is a perspective view showing the bottom of the apparatus of FIG. 18 comprising a still further exemplary cover,

FIG. 20 is an enlarged view showing a guide hole member of the covers of FIGS. 18 and 19,

FIG. 21 shows electrode devices and electrical lines of an apparatus according to the first embodiment of the present invention,

FIG. 22 is a perspective view of an apparatus for measuring impedance of a human body according to the second embodiment of the present invention,

FIG. 23 is a perspective view of the bottom of the apparatus of FIG. 22,

FIG. 24 shows electrode devices and electrical lines of the apparatus of FIG. 22,

FIG. 25 is a plane view of an apparatus according the third embodiment of the present invention, and

FIG. 26 is a plane view of an apparatus according the fourth embodiment of the present invention.

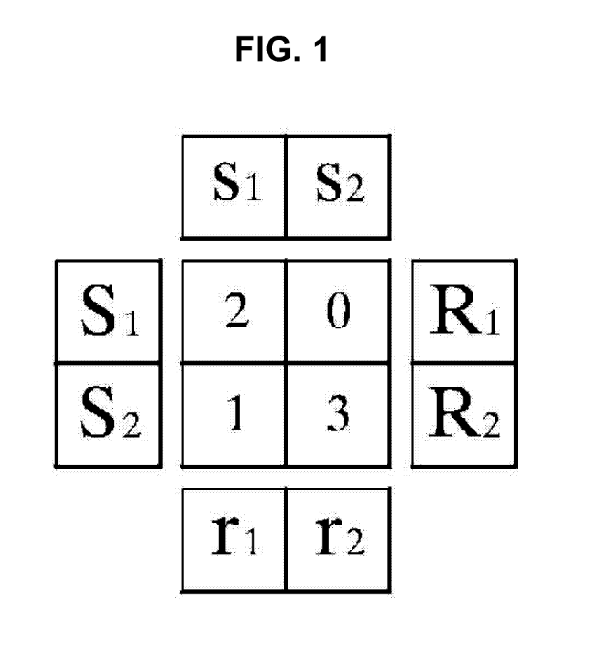

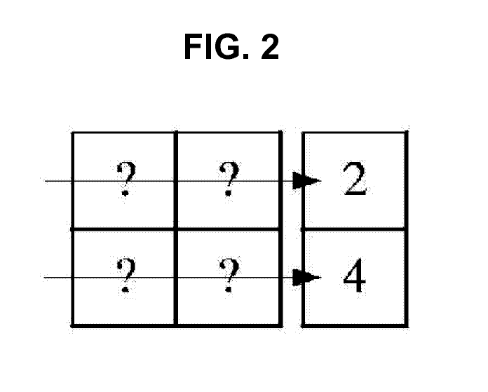

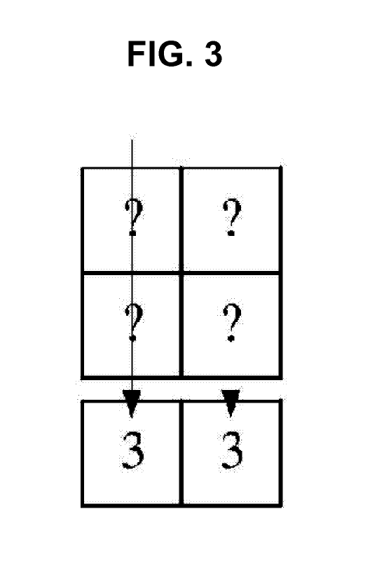

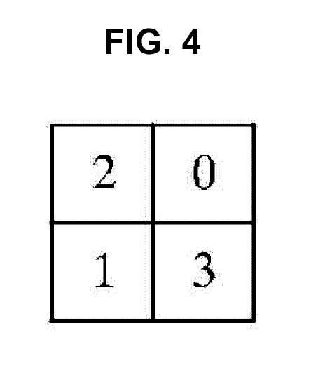

### DETAILED DESCRIPTION OF EMBODIMENTS

Reference will now be made in detail to the preferred embodiment of the present invention, examples of which are illustrated in the drawings attached hereinafter, wherein like reference numerals refer to like elements throughout. The embodiments are described below so as to explain the present invention by referring to the figures.

Terms used herein are those used generally in the art unless otherwise being defined herein, in which case such terms are described in the specification and they should be construed by replying on the description. Also, detailed description of the subject matter that is known to those skilled in the art and is not directly related to the present invention is omitted for simplicity.

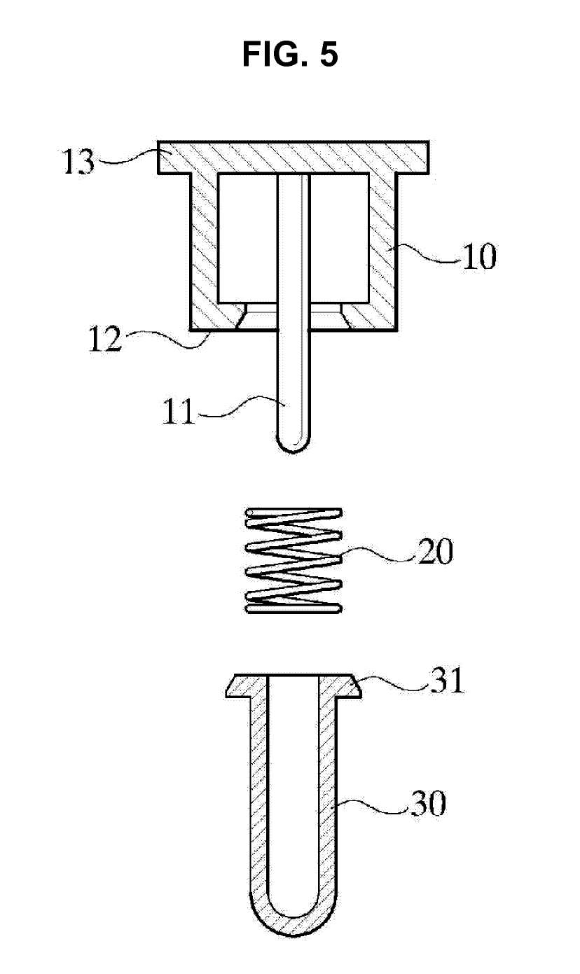

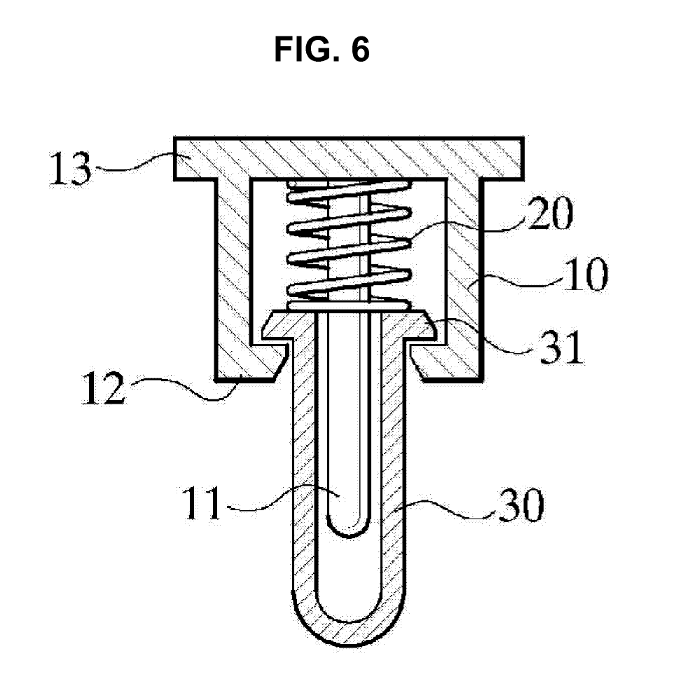

FIG. 5 is a cross-sectional view showing a dissembled state of an electrode device for measuring impedance of a human body according to the first embodiment of the present invention and FIG. 6 is a cross-sectional view showing an assembled state of the electrode device of FIG. 5.

The electrode device according to the first embodiment, as shown in FIGS. 5 and 6, comprises a cylindrical housing member (10), a cylindrical electrode member (20), and a resilient member (30). The cylindrical housing member (10) has a guide rod (11) mounted to an open side thereof. The cylindrical electrode member (30) is configured to be pressed against the skin of a human body to move back and forth through the open side along the guide rod (11). The resilient member (30) is interposed between the cylindrical electrode member (20) and the cylindrical housing member (10) to resiliently move the cylindrical electrode member (20) back and forth.

The cylindrical electrode member (30) can be an electrode made of a conductive material. It can also be an electrode coated with a conductive material. Any material that shows conductivity can be used as the conductive material. Preferably, the conductive material is non-toxic to a human body. As an example, an electrode made of a gold or an electrode coated with gold can be used. The cylindrical electrode member (30) has an open end. A protruding portion (31) is formed at the open end of the cylindrical electrode member (30). The protruding portion (31) may be formed in any shape that can facilitate engagement between the cylindrical electrode member (30) and the cylindrical housing member (10).

The cylindrical housing member (10) can be made of a conductive material. It can also be an electrode coated with a conductive material. Any material that shows conductivity can be used as the conductive material. As an example, an electrode made of gold or an electrode coated with gold can be used. Also, a copper wire or an iron wire can be used. A first protruding portion (12) is formed at the open side of the cylindrical housing member (10). The first protruding portion (12) is formed in any shape that can facilitate engagement between the cylindrical electrode member (30) and the cylindrical housing member (10). A second protruding portion (13) is formed at the other side of the cylindrical housing member (10).

The resilient member (20) can be made of any material that can show resilient features. The resilient material can also be a conductive material. As an example, a spring can be used.

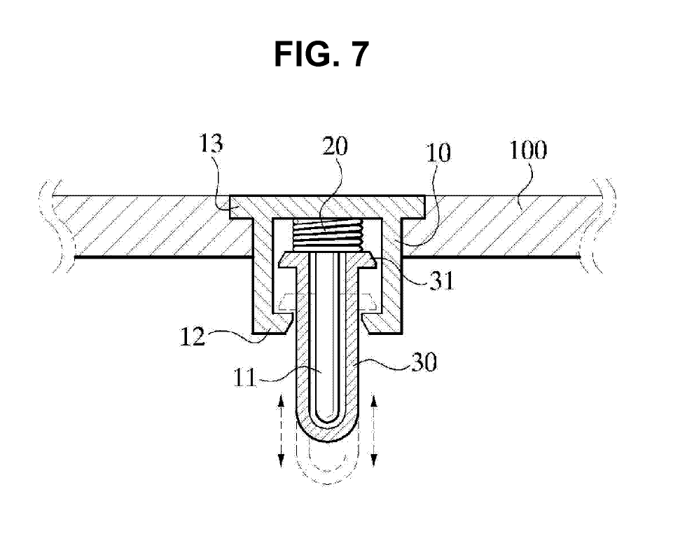

FIG. 7 is a cross-sectional view showing an example of application of the electrode device of FIG. 5. As shown in FIG. 7, when the cylindrical electrode member (30) is pressed against the skin of a human body, the cylindrical electrode member (30) can be moved along the guide rod by the resilient member (20) according to the curvature of the skin. Accordingly, the depths of the cylindrical electrode members that are attached to the skin can be set to be a certain value. This can avoid a prior art problem that impedance was not be able to be measured precisely since the depths of electrode devices attached to the skin are not constant. The reference number 100 refers to a base plate in which electrode devices according to the present invention are mounted.

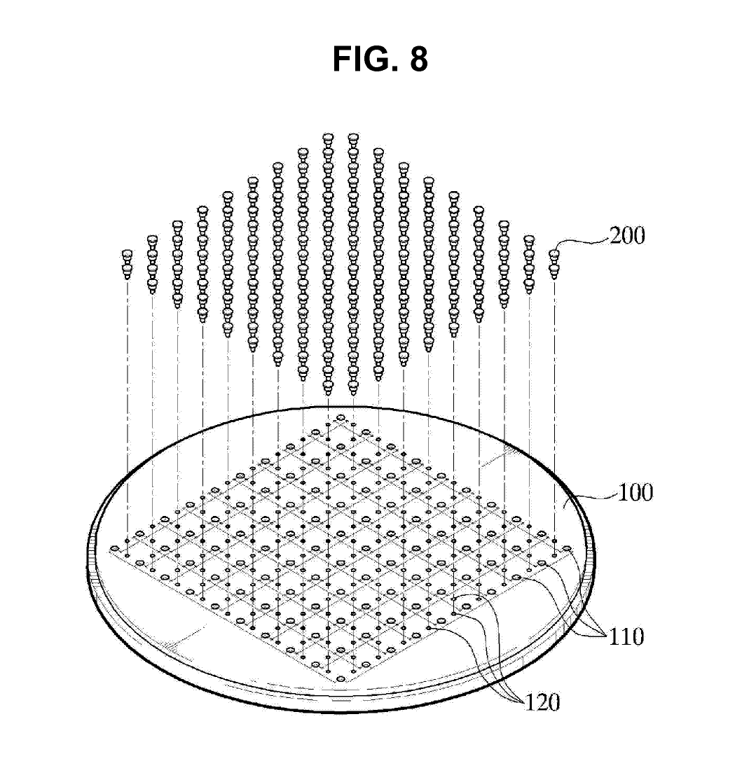

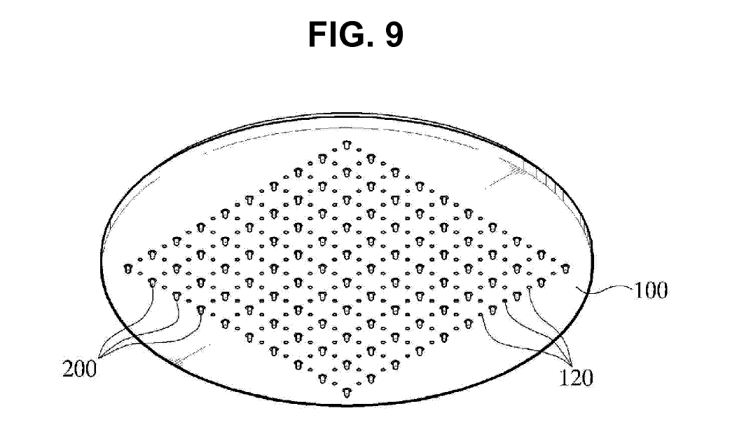

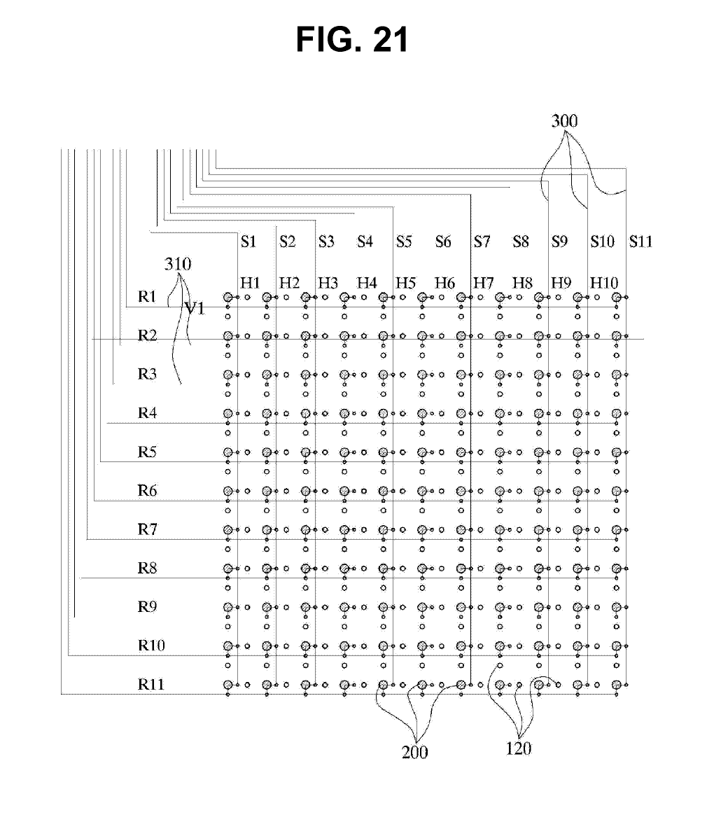

FIG. 8 is a perspective view of an apparatus for measuring impedance of a human body according to a first embodiment of the present invention, and FIG. 9 is a perspective view of the bottom of the apparatus of FIG. 8. As shown in FIGS. 8 and 9, the apparatus according to the first embodiment comprises a base plate (100) and a plurality of electrode devices (200). The base plate (100) has a plurality of electrode holes (110) and a plurality of first needle holes (120). The electrode holes (100) are arranged in an X-Y lattice structure. The first needle holes (120) are arranged in an X-Y lattice structure such that they are arranged with the electrode holes (100) alternately. The electrode devices each are mounted in the respective electrode holes (110). The electrode devices are connected to a plurality of first and second electrical lines (300, 310), which will be described in detail with reference to FIG. 21. The base plate (100) can be made of any soft material that can make the base plate curved according to the curvature of the skin of a human body. As an example, it can be made of silicone.

The distance between two adjacent electrode holes and the distance between two adjacent first needle holes are not limited to a specific value. Preferably, the distances can be set in a range of 5 mm to 20 mm.

The width of the first needle holes each is not limited to a specific value. It can be appropriately set according to the kind of a needle and/or the purpose of acupuncture.

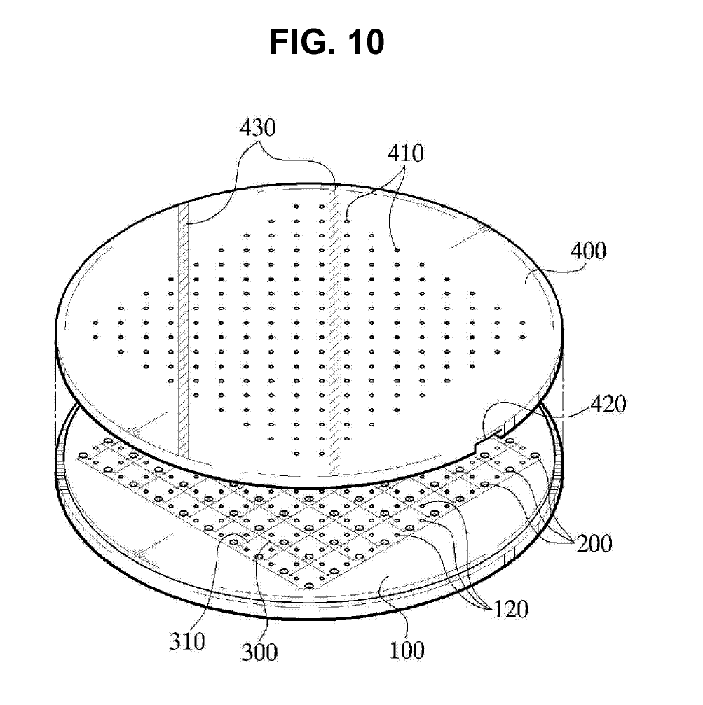

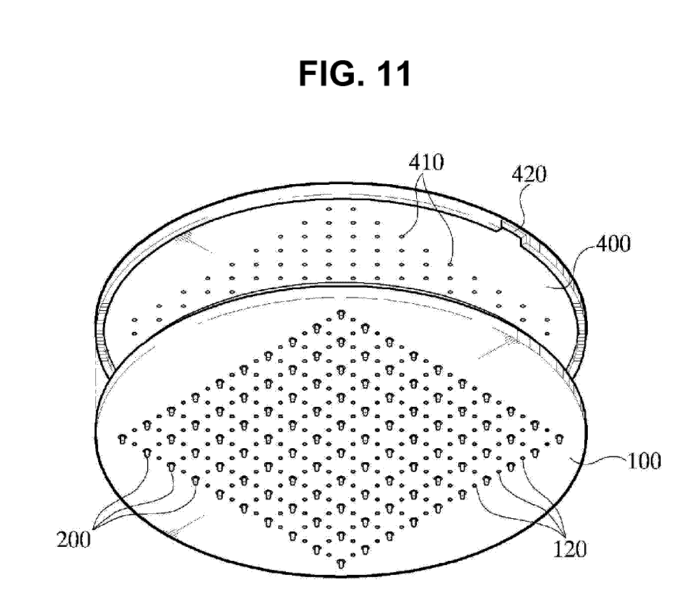

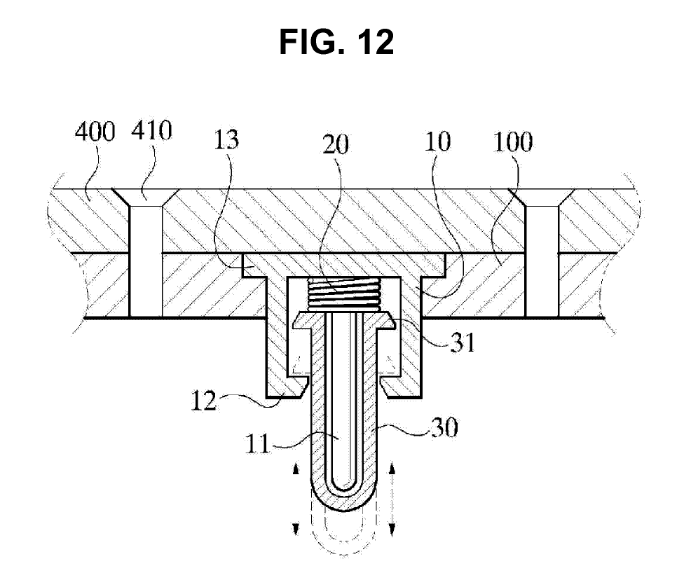

FIG. 10 is a perspective view showing the apparatus of FIG. 8 comprising an exemplary cover and FIG. 11 is a perspective view showing the bottom of the apparatus comprising the cover. The exemplary cover (400), as shown in FIGS. 10 and 11, is configured to be engaged with the base plate (100) to protect the electrode devices (200) and the first and second electrical lines (300, 310). The cover (400) has a plurality of second needle holes (410). The second needle holes (410) are arranged in the cover (400) such that the respective second needle holes (410) can be positioned over the respective first needle holes (120) of the base plate (100) when the cover (400) is engaged with the base plate (100). The second needle holes can be designed to have the same width as the first needle holes. Preferably, as shown in FIG. 12, the second needle holes (410) can be designed such that the ourter width is greater than the inner width, which makes it easier for a user to insert a needle to the second needle holes (410). In some embodiments, at least one identifying member (430) may be provided on the cover (400) to make it easier for a user to handle the cover (400). The cover (400) can be made of any material that can perform the above-described function. As an example, it can be made of silicone. The cover (400) is provided with a connecting hole (420) through which electrical lines can be connected.

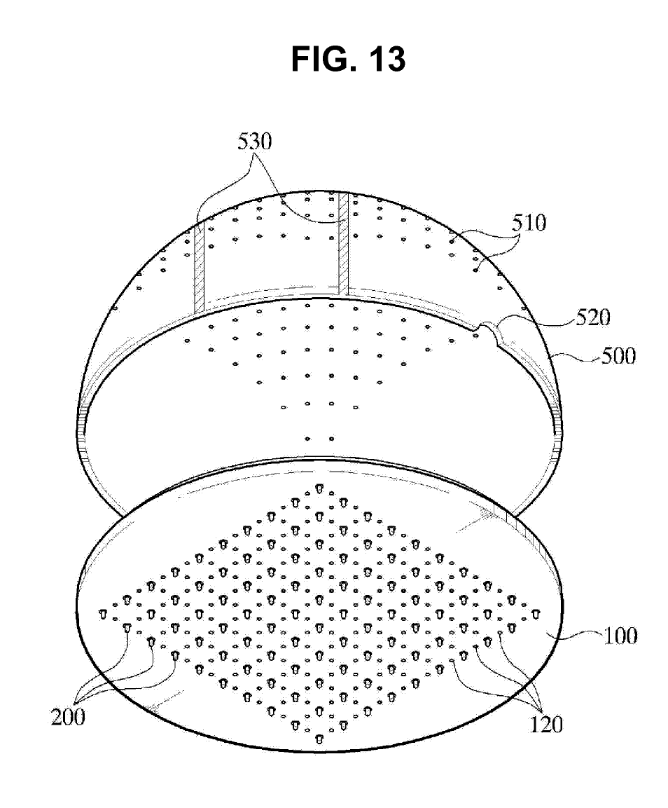

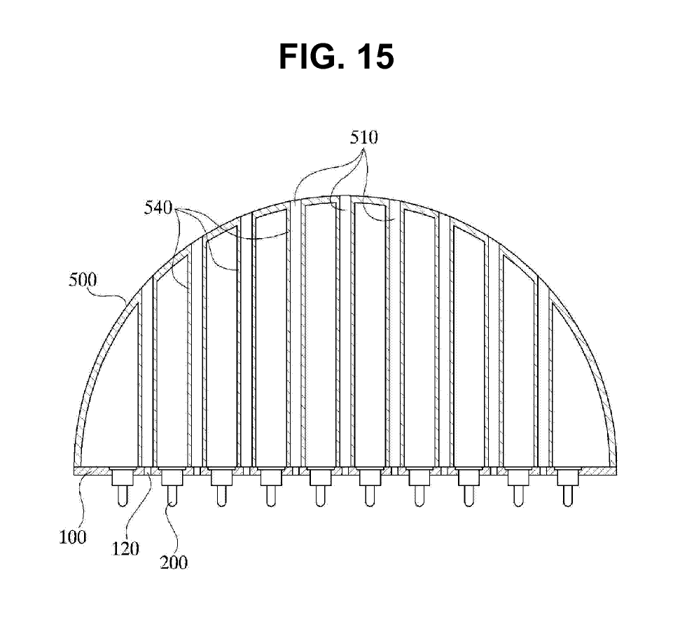

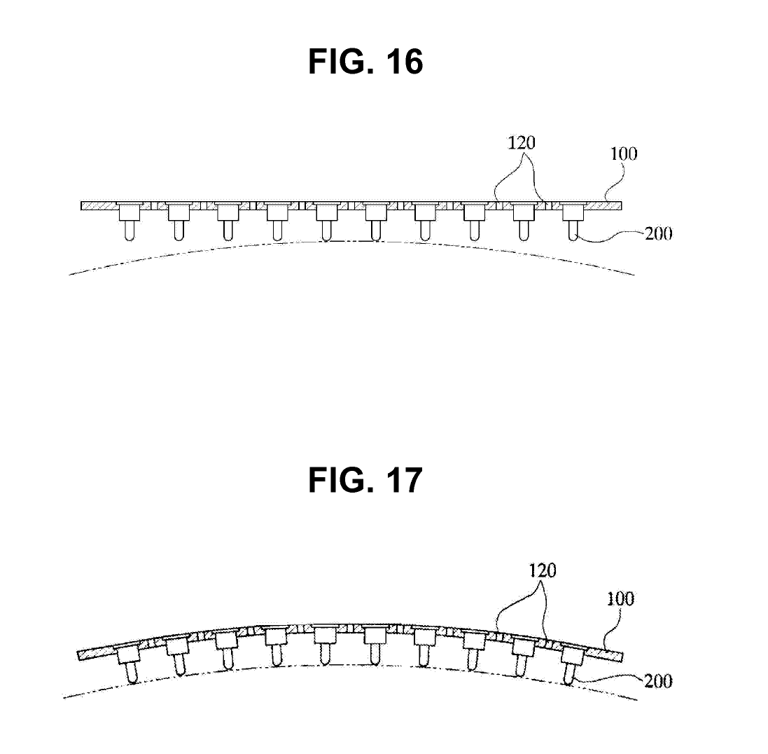

FIG. 13 is perspective view showing the apparatus of FIG. 8 comprising another exemplary cover. As shown in FIG. 13, the cover (500) is in the form of a hemisphere. The cover (500) has a plurality of second needle holes (510). The second needle holes (510) are arranged in the cover (500) such that the respective second needle holes (510) can be positioned over the respective first needle holes (120) of the base plate (100) when the cover (500) is engaged with the base plate (100). In some embodiments, the base plate (100) is designed to be flexible so as to become curved according to the curvature of the skin, as shown in FIG. 17 while the base plate (100) is flat in normal condition, as shown in FIG. 16. In some embodiments, as shown in FIG. 15, the cover (500) has a plurality of guide hole members (540) each connecting the first needle holes (120) and the second needle holes (510) to make it easier for a user to handle a needle. As described above, preferably, the second needle holes (510) can be designed such that the outer width is greater than the inner width, which makes it easier for a user to insert a needle to the second needle holes (510). The cover (500) can be made of any material that can perform the above-described function. As an example, it can be made of silicone. The cover (500) is provided with a connecting hole (520) through which electrical lines can be connected. In some embodiments, at least one identifying member (530) may be provided on the cover (500) to make it easier for a user to handle the cover (500).

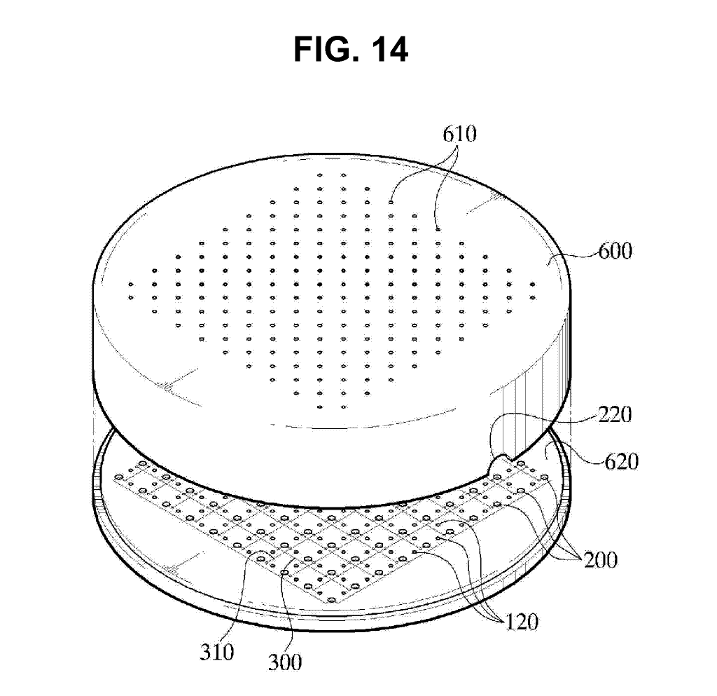

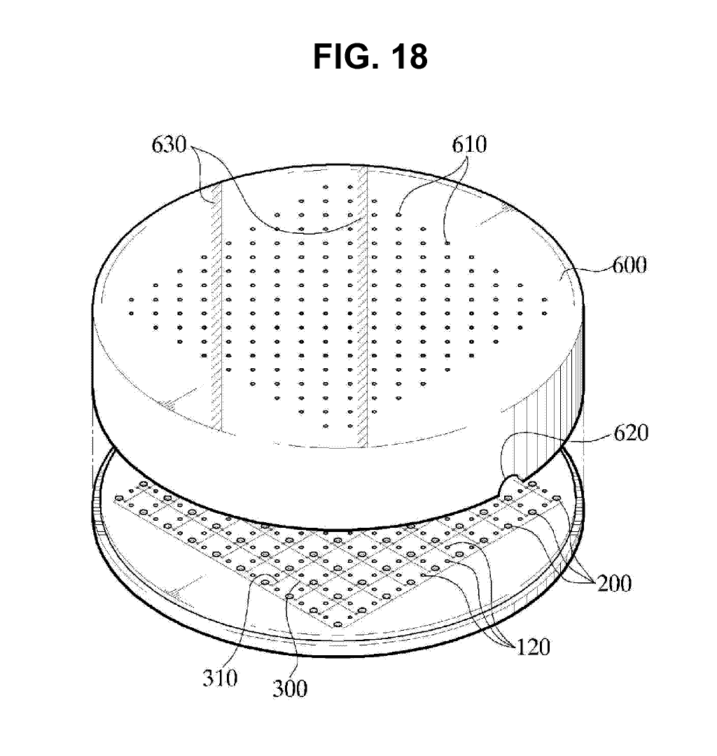

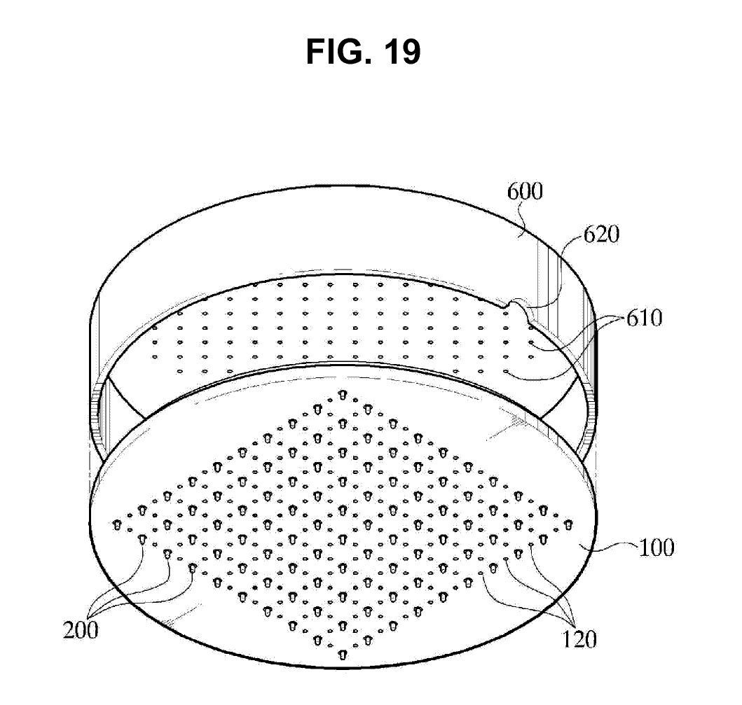

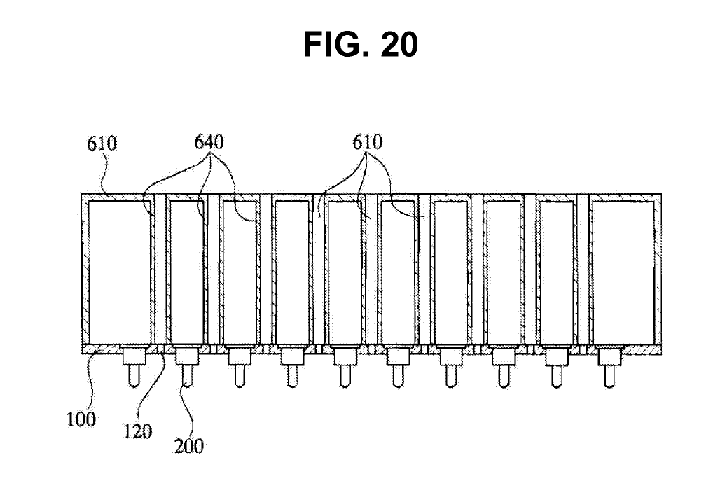

FIGS. 14 and 18 are perspective views showing the apparatus of FIG. 8 comprising another exemplary cover and FIG. 19 is a perspective view showing the bottom of the apparatus comprising the cover shown in FIG. 18. As shown in the drawings, the cover (600) is in the form of a cylinder. The cover (600) has a plurality of second needle holes (610). The second needle holes (610) are arranged in the cover (600) such that the respective second needle holes (610) can be positioned over the respective first needle holes (120) of the base plate (100) when the cover (600) is engaged with the base plate (100). In some embodiments, the base plate (100) is designed to be flexible so as to become curved according to the curvature of the skin, as shown in FIG. 17 while the base plate (100) is flat in normal condition, as shown in FIG. 16. In some embodiments, as shown in FIG. 20, the cover (500) has a plurality of guide hole members (640) connecting the first needle holes (120) and the second needle holes (610) to make it easier for a user to handle a needle. As described above, preferably, the second needle holes (610) can be designed such that the outer width is greater than the inner width, which makes it easier for a user to insert a needle to the second needle holes (610). The cover (600) can be made of any material that can perform the above-described function. As an example, it can be made of silicone. The cover (600) is provided with a connecting hole (620) through which electrical lines can be connected. In some embodiments, at least one identifying member (630) may be provided on the cover (600) to make it easier for a user to handle the cover (600).

FIG. 21 shows electrode devices and electrical lines of an apparatus according to the first embodiment of the present invention. The electrode devices (200) and the first needle holes (120) are positioned on the base plate (100) such that they are alternating with each other. The first electrical lines (300) are formed between the respective electrode devices (200) and the respective first needle holes (120) in the direction of X-axis. The second electrical lines (310) are formed between the respective electrode devices (200) and the respective first needle holes (120) in the direction of Y-axis.

The respective first and second electrical lines (300, 310) are connected to the respective electrode devices (200). Current is allowed to flow through the X-axis and Y-axis sequentially to measure impedances. For example, with the second vertical line S2 as an input electrode and the first horizontal line R1 as a receiving electrode, the impedance of the first horizontal needle hole H1 can be measured. Also, with the first vertical line S1 as an input electrode and the second horizontal line R2 as a receiving electrode, the impedance of the first vertical needle hole V1 can be measured. Accordingly, if current is allowed to flow from the first vertical line S1 to the eleventh vertical line S11 sequentially to act as input electrodes and from the horizontal line R1 to the eleventh horizontal line R11 to act as receiving electrodes, impedances can be measured sequentially with regard to all of the vertical and horizontal needle holes of the base plate (100). The measured impedances can be displayed as a 3-dimensional image by being coupled with CT or MRI devices.

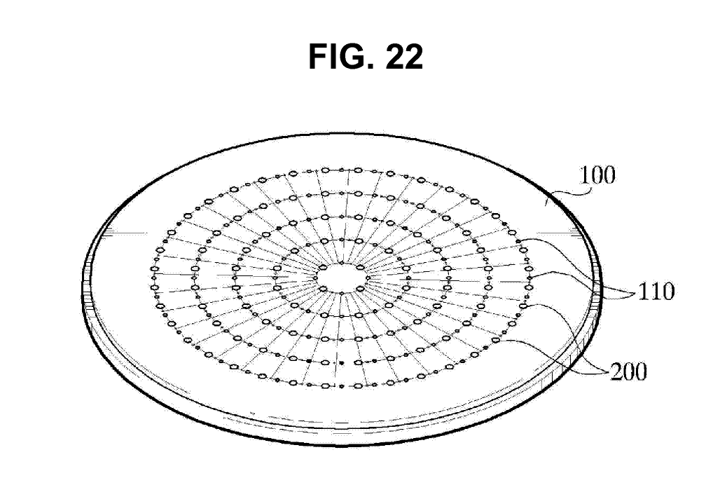

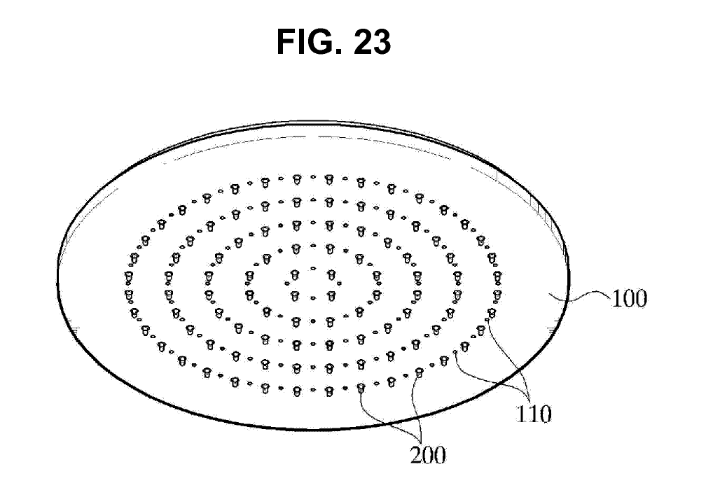

FIG. 22 is a perspective view of an apparatus according to the second embodiment of the present invention and FIG. 23 is a perspective view of the bottom of the apparatus of FIG. 22. As shown in FIGS. 22 and 23, the apparatus according to the second embodiment comprises a circular base plate (100) and a plurality of electrode devices (200). The circular base plate (100) includes a plurality of electrode hoes (110) and a plurality of first needle holes (120). The electrode holes (110) and first needle holes (120) are formed eccentrically and alternately, as shown in FIG. 22. The electrode devices (200) are mounted to the electrode holes (110) and connected to a first and second electrical lines (300, 310)

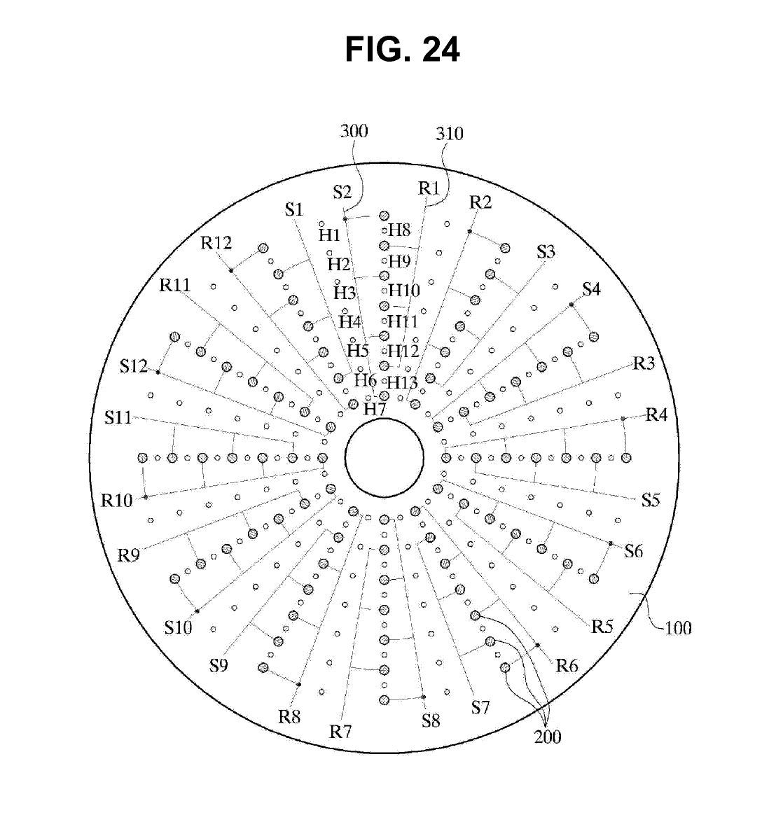

FIG. 24 shows electrode devices and electrical lines of the apparatus of FIG. 22. The first and second electrical lines (300, 310) are connected to the electrode devices alternately. Current is allowed sequentially to measure impedances. For example, with the second input line S2 as an input electrode and the twelveth receiving line R12 as a receiving electrode, the impedances of the first, third, fifth, and seventh needle holes H1, H3, H5, and H5 can be measured. Also, with the first input line S1 as an input electrode and the first receiving line R1 as a receiving electrode, the impedances of the second, fourth, and sixth needle holes H2, H4, and H6 can be measured. Accordingly, if current is allowed to flow from the first input line S1 to the twelveth input line S12 and from the first receiving line R1 to the twelveth receiving line R12 sequentially to act as input electrodes and receiving electrodes, respectively, the impedances can be measured sequentially with regard to all of needle holes of the base plate (100).

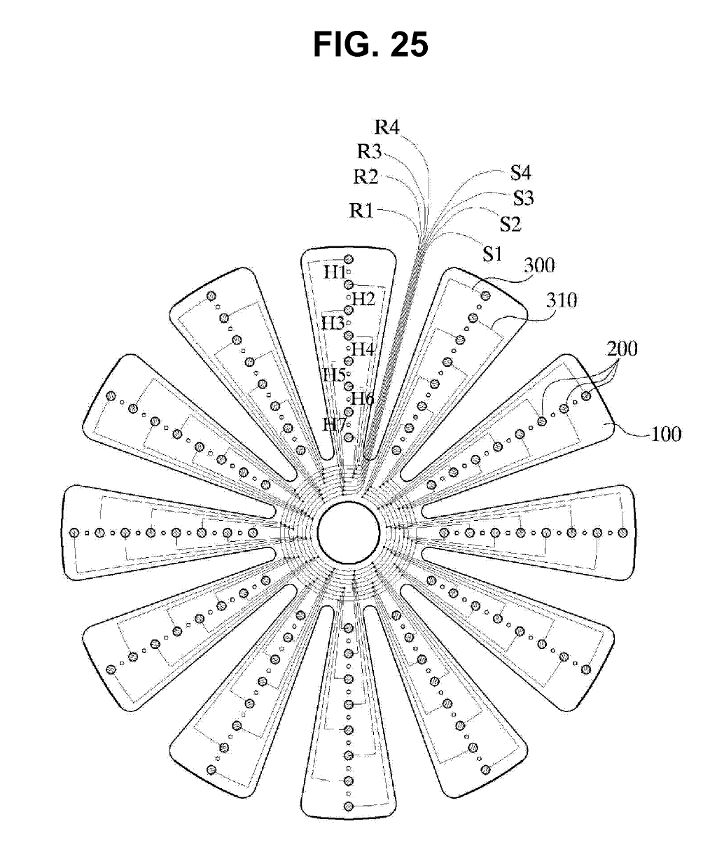

FIG. 25 is a plane view of an apparatus according the third embodiment of the present invention. In this embodiment, a plurality of base plates (100) are provided radially with a predetermined distance between two adjacent base plates (100). Each of the base plates has a plurality of electrode holes and a plurality of first needle holes (120). The electrode holes and the first needle holes are mounted alternately. Current is allowed sequentially to measure impedances. For example, with the first input line S1 as an input electrode and the first receiving line R1 as a receiving electrode, the impedance of the first needle hole H1 can be measured. Also, with the second input line S2 as an input electrode and the first receiving line R1 as a receiving electrode, the impedance of the second needle hole H2 can be measured. Accordingly, if current is allowed to flow sequentially, the impedances can be measured sequentially with regard to all of needle holes of the base plate (100).

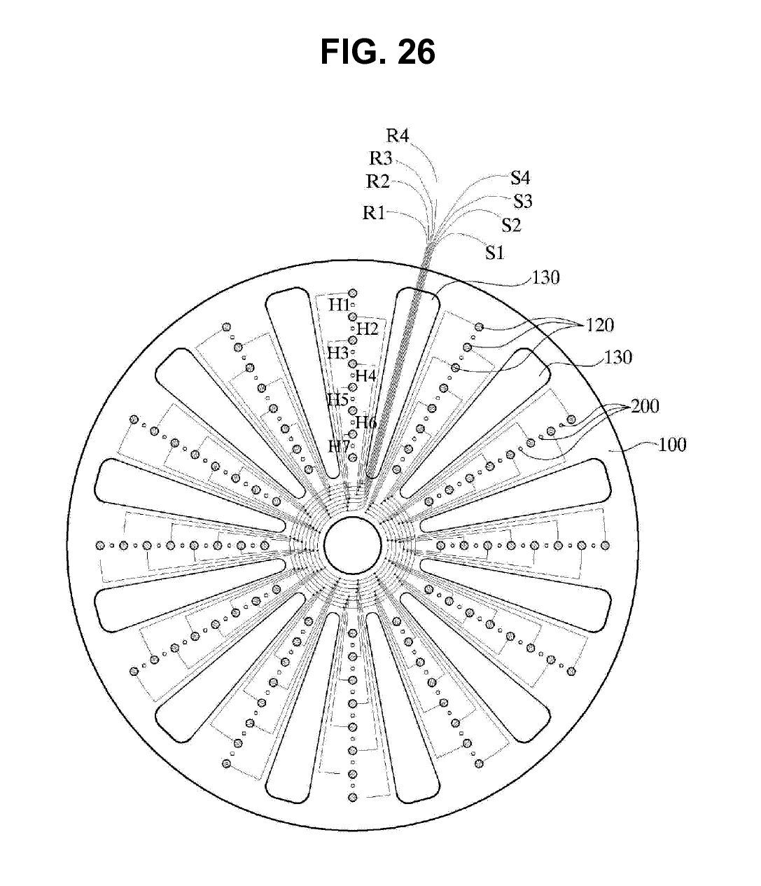

FIG. 26 is a plane view of an apparatus according the fourth embodiment of the present invention. In this embodiment, the apparatus comprises a base plate, a plurality of electrode devices, and first and second electrical lines. The base plate has a plurality of radial holes provided radially with a predetermined distance between two adjacent holes. The base plate has a plurality of electrode holes and a plurality of first needle holes between two adjacent radial holes. The electrode holes and the first needle holes are mounted alternately. The electrode devices are mounted to the electrode holes. The first and second electrical lines are connected to the electrode devices alternately. As the principle of measuring impedances is similar to the principle described with regard to FIG. 25, detailed description thereof is omitted.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

1. An electrode device for measuring impedances of a human body, comprising:
a cylindrical housing member having a guide rod mounted to an open side thereof;
a cylindrical electrode member configured to be pressed against the skin of a human body to move back and forth through the open side along the guide rod; and
a resilient member interposed between the cylindrical electrode member and the cylindrical housing member to resiliently move the cylindrical electrode member back and forth along the guide rod.

2. The electrode device of claim 1, wherein the cylindrical electrode member is an electrode made of a conductive material or an electrode coated with a conductive material.

3. The electrode device of claim 2, wherein the conductive material is gold.

4. The electrode device of claim 1, wherein a protruding portion is formed at the open end of the cylindrical electrode member such that engagement between the cylindrical electrode member and the cylindrical housing member is facilitated.

5. The electrode device of claim 1, wherein the cylindrical housing member is made of a conductive material or an electrode coated with a conductive material.

6. The electrode device of claim 1, wherein a first protruding portion is formed at the open side of the cylindrical housing member and a second protruding portion is formed at the other side of the cylindrical housing member.

7. The electrode device of claim 1, wherein the resilient member is made of a conductive material.

8. The electrode device of claim 7, wherein the resilient member is a spring.

9. An apparatus for measuring impedance of a human body, which comprises:
a base plate having a plurality of electrode holes formed in a lattice structure and a plurality of a first needle holes formed in a lattice structure such that the electrode holes and the first needle holes are arranged alternately;
a plurality of electrode devices mounted to the electrode holes; and
first and second electrical lines connected to the electrode devices.

10. The apparatus of claim 9, wherein the distance between two adjacent electrode holes and the distance between two adjacent first needle holes are set in a range of 5 mm to 20 mm.

11. The apparatus of claim 9, further comprising a cover configured to be engaged with the base plate to protect the electrode devices and the first and second electrical lines.

12. The apparatus of claim 10, wherein the cover includes a plurality of second needle holes arranged such that the respective second needle holes can be positioned over the respective first needle holes of the base plate when the cover is engaged with the base plate.

13. The apparatus of claim 12, wherein the outer width of the second needle holes is greater than the inner width thereof.

14. The apparatus of claim 10, wherein the cover is provided with at least one identifying member.

15. The apparatus of claim 10, wherein the cover is made of silicone.

16. The apparatus of claim 11, wherein the cover is shaped to be a hemisphere or a cylinder.

17. The apparatus of claim 16, wherein the cover further comprises a plurality of guide hole members each connecting the first needle holes and the second needle holes.

18. The apparatus of claim 9, wherein the first electrical lines are formed between the respective electrode devices and the respective first needle holes in the direction of X-axis and the second electrical lines are formed between the respective electrode devices and the respective first needle holes in the direction of Y-axis.

19. The apparatus of claim 9, wherein the first electrical lines are connected to input electrodes and the second electrical lines are connected to receiving electrodes.

20. An apparatus for measuring impedances of a human body, comprising:
a circular base plate including a plurality of electrode hoes and a plurality of first needle holes, the electrode holes and the first needle holes being formed eccentrically and alternately;
a plurality of electrode devices mounted to the electrode holes; and
first and second electrical lines connected to the electrode devices.

21. The apparatus of claim 20, wherein the first electrical lines and the second electrical lines are connected to the electrode devices alternately.

22. An apparatus for measuring impedances of a human body, comprising:
a plurality of base plates provided radially with a predetermined distance between two adjacent base plates, each of which base plates has a plurality of electrode holes and a plurality of first needle holes, the electrode holes and the first needle holes being mounted alternately;
a plurality of electrode devices mounted to the electrode holes; and
first and second electrical lines connected to the electrode holes alternately.

23. An apparatus for measuring impedances of a human body, comprising:
a plurality of base plates provided radially with a predetermined distance between two adjacent base plates, each of which base plates has a plurality of electrode holes and a plurality of first needle holes, the electrode holes and the first needle holes being mounted alternately with respect to the center of base plates;
a plurality of electrode devices mounted to the electrode holes; and
first and second electrical lines connected to the electrode holes alternately.

24. An apparatus for measuring impedances of a human body, comprising:
a base plate having a plurality of radial holes provided radially with a predetermined distance between two adjacent holes, the base plate having a plurality of electrode holes and a plurality of first needle holes between two adjacent radial holes;
a plurality of electrode devices mounted to the electrode holes; and
first and second electrical lines connected to the electrode devices alternately,
wherein the electrode holes and the first needle holes are mounted alternately.

25. The apparatus of claim 22, wherein the first electrical lines are connected to input electrodes and the second electrical lines are connected to receiving electrodes.

26. The apparatus of claim 23, wherein the first electrical lines are connected to input electrodes and the second electrical lines are connected to receiving electrodes.

27. The apparatus of claim 24, wherein the first electrical lines are connected to input electrodes and the second electrical lines are connected to receiving electrodes.
